(19) United States
(12) Patent Application Publication
(10) Pub. No.: US 2024/0391867 A1
(43) Pub. Date: Nov. 28, 2024

(54) UREA PRODUCTION DEVICE AND UREA PRODUCTION METHOD

(71) Applicants: NATIONAL UNIVERSITY CORPORATION TOKAI NATIONAL HIGHER EDUCATION AND RESEARCH SYSTEM, Nagoya-shi (JP); SAWAFUJI ELECTRIC CO., LTD., Ota-shi (JP); KAWADA INDUSTRIES, INC., Tokyo (JP)

(72) Inventors: Shinji KAMBARA, Gifu (JP); Tomonori Miura, Gunma (JP); Yuya Tanaka, Gunma (JP); Shigeo Takitani, Toyama (JP); Tomokazu Kodera, Toyama (JP)

(21) Appl. No.: 18/796,708

(22) Filed: Aug. 7, 2024

Related U.S. Application Data
(63) Continuation of application No. PCT/JP2023/002846, filed on Jan. 30, 2023.

(30) Foreign Application Priority Data
Feb. 8, 2022 (JP) 2022-017752

(51) Int. Cl. C07C 273/04

(52) U.S. Cl. CPC C07C 273/04

(57) ABSTRACT

A urea production device includes: a body portion; a first electrode disposed inside the body portion; and a second electrode which is disposed such that at least a part thereof faces the first electrode. The urea production device includes a gas flow path formed between the body portion and either the first electrode or the second electrode, a first raw material introduction path having one side connected to the gas flow path and another side connected to a carbon dioxide storage source, and a second raw material introduction path which is a raw material introduction path different from the first raw material introduction path and which has one side connected to the gas flow path and another side connected to an ammonia storage source. A voltage is to be applied between the first electrode and the second electrode and electric discharge can be generated.

### CROSS-REFERENCE TO RELATED APPLICATIONS

This application is a continuation of PCT application No. PCT/JP2023/002846, which was filed on Jan. 30, 2023 based on Japanese Patent Application (No. P2022-017752) filed on Feb. 8, 2022, the contents of which are incorporated herein by reference.

### TECHNICAL FIELD

The present invention relates to a urea production device and a urea production method.

### BACKGROUND

In order to achieve carbon neutrality, which means reducing overall greenhouse gas emissions to zero, it is required to establish a technology to fix carbon dioxide and utilize the carbon dioxide as a resource. For example, studies have been made to separate and collect carbon dioxide from exhaust gas emitted from thermal power plants or the like and produce useful chemical substances from the collected carbon dioxide. Some technologies related to carbon dioxide separation and collection have already reached the stage of practical application. On the other hand, there is still room for improvement in many technologies for converting carbon dioxide into useful substances and utilizing the carbon dioxide.

Urea is one of the substances that can be produced using carbon dioxide as a raw material. Urea can be used as a raw material for chemical products, medicine, and fertilizer, and is an excellent carbon dioxide fixing substance that does not re-emit carbon dioxide.

At present, a widespread method for producing urea is a direct synthesis method in which carbon dioxide and ammonia are directly reacted. For example, as a related art, Patent Literature 1 discloses that the yield of urea is 50% to 60% at a temperature of 160°° C. to 250° C. and a pressure of 40 MPa, and the yield of urea is 20% to 34% at a temperature of 150° C. to 200° C. and a pressure of 8.2 MPa to 12.4 MPa. In addition, Patent Literature 1 discloses, as a method for producing a urea compound, a synthesis method using water in which carbon dioxide is dissolved as a reaction medium, a water-soluble salt as a catalyst, and an amine compound represented by the formula R—NH₂ as a raw material for the amine compound. In a first embodiment of Patent Literature 1, a technology is disclosed in which carbon dioxide is dissolved in a sodium carbonate aqueous solution at a pressure of 5 MPa and reacted with decylamine at a temperature of 180° C. to produce didecyl urea, and as a result, the dissolved carbon dioxide is fixed at 100%.

As disclosed in Patent Literature 1, the production of urea and the urea compound in the related art is effective as a technology for fixing carbon dioxide. However, the production of urea in the related art needs to be performed under high-temperature and high-pressure conditions.
Patent Literature 1: JP2012-111711A

The present invention has been made in view of the above circumstances, and an object of the present invention is to provide a urea production device and a urea production method capable of producing urea using carbon dioxide and ammonia as raw materials under conditions that do not require high temperature or high pressure.

### SUMMARY

The present invention relates to a urea production device. The urea production device of the present invention is a urea production device including: a body portion including a dielectric; a first electrode disposed inside the body portion; and a second electrode which is external to the body portion and which is disposed such that at least a part thereof faces the first electrode, in which the urea production device includes a gas flow path formed between the body portion and either the first electrode or the second electrode, a first raw material introduction path having one side connected to the gas flow path and another side connected to a carbon dioxide storage source, and a second raw material introduction path which is a raw material introduction path different from the first raw material introduction path and which has one side connected to the gas flow path and another side connected to an ammonia storage source, and a voltage is to be applied between the first electrode and the second electrode and electric discharge can be generated.

The urea production device of the present invention can produce urea by applying a voltage between the first electrode and the second electrode disposed inside and outside the gas flow path to generate electric discharge, and by converting carbon dioxide and ammonia introduced into the gas flow path into plasma and reacting the carbon dioxide and the ammonia.

Preferably, the gas flow path is formed between the body portion and the first electrode, and a distance between an outer peripheral surface of the first electrode and an inner peripheral surface of the body portion is 0.1 mm or more and 5 mm or less.

Preferably, the gas flow path is formed between the body portion and the second electrode, and a distance between an inner peripheral surface of the second electrode and an outer peripheral surface of the body portion is 0.1 mm or more and 5 mm or less.

Preferably, the first raw material introduction path and the second raw material introduction path are connected to one end portion of the gas flow path, and another end portion of the gas flow path is provided with a collection part configured to collect urea produced by a synthesis reaction of ammonia and carbon dioxide due to the electric discharge.

The present invention also provides a urea production method. The urea production method of the present invention is a urea production method in which urea is produced by generating electric discharge between a first electrode disposed inside a body portion including a dielectric and a second electrode disposed outside the body portion and converting carbon dioxide and ammonia flowing into a gas flow path formed between the body portion and either the first electrode and the second electrode into plasma, the urea production method including: a step of introducing the carbon dioxide into the gas flow path; a step of converting the carbon dioxide into the plasma by generating the electric discharge between the first electrode and the second electrode; a step of introducing the ammonia into the gas flow path while the electric discharge continues; and a step of converting the ammonia into the plasma by generating the electric discharge.

Preferably, in the urea production method of the present invention, a molar ratio of the carbon dioxide and the ammonia to be introduced into the gas flow path is the carbon dioxide:the ammonia=1:1 or more and 1:3 or less.

As described above, the urea production device and the urea production method of the present invention can produce urea using carbon dioxide and ammonia as raw materials under conditions that do not require high temperature or high pressure.

### BRIEF DESCRIPTION OF DRAWINGS

FIG. 1 is a diagram schematically showing a vertical cross-section view of a preferable urea production device of the present invention.

FIG. 2 is a graph showing a relation between a molar ratio of ammonia and carbon dioxide supplied to the urea production device of the present invention and the amount of urea produced.

FIG. 3 is a graph showing a relation between a ratio of the ammonia in a raw material supplied to the urea production device of the present invention and a conversion rate of the carbon dioxide.

FIG. 4 is a diagram showing an analysis result of a substance produced by the urea production device of the present invention by proton nuclear magnetic resonance.

FIG. 5 is a diagram showing an analysis result of the substance produced by the urea production device of the present invention by Fourier transform infrared ray spectroscopy.

FIG. 6 is a vertical cross-section view of a urea production device according to another embodiment of the present invention.

FIG. 7 is a flowchart of a preferred urea production method of the present invention.

FIG. 8 is a vertical cross-section view schematically showing the urea production device according to the present embodiment.

### DESCRIPTION OF EMBODIMENTS

Hereinafter, preferred embodiments of a urea production device and a urea production method of the present invention will be listed.
(1) The container of the urea production device includes a body member (body portion in the present invention) including a cylindrical dielectric, and sealing members that hold a first electrode inside the body member.
(2) The dielectric of the body member can be made of ceramics such as alumina, highly insulating resins such as barium titanate, polycarbonate, and acrylic, glasses such as quartz glass, and combinations of these materials. The most preferable material for the dielectric is quartz glass.
(3) A preferred sealing member is an O-ring made of silicone.
(4) The first electrode has a rod shape with a circular cross section, and an entire length thereof is longer than that of the body member of the container. Recesses for accommodating a part of the sealing members are formed at two end portions of the first electrode. The first electrode is accommodated inside the body member in a state in which the sealing members are engaged with the recesses, thereby being concentrically fixed to the body member.
(5) The sealing members close portions between an outer peripheral surface of the first electrode and an inner peripheral surface of the body member of the container. A gas flow path having a distance of 0.1 mm or more and 5 mm or less is formed between the body member and the first electrode.
(6) A second electrode is disposed at a position facing the first electrode while being in contact with an outer peripheral surface of the body member. In the gas flow path between the first electrode and the second electrode, a synthesis reaction of ammonia and carbon dioxide to be described later occurs.

7) The first electrode is fixed in such a manner that the end portions on two sides protrude outward from the body member.
(8) A first raw material introduction path connecting a carbon dioxide storage source and the gas flow path and a second raw material introduction path connecting an ammonia storage source and the gas flow path are connected to one end portion of the gas flow path. The first raw material introduction path and the second raw material introduction path are different (independent) flow paths and can allow the ammonia and the carbon dioxide to flow into the gas flow path without mixing the ammonia and the carbon dioxide.
(9) A gas discharge path connected to a collection part is connected to another end portion of the gas flow path.
(10) A cooling tank and a collection part are provided at an outlet of the gas discharge path, and urea and excess ammonia discharged in a urea production stage can be mainly collected. In addition, the collection part is cooled by the cooling tank and can collect the urea more efficiently.
(11) In the urea production method, first, carbon dioxide is supplied from the carbon dioxide storage source to the gas flow path, the carbon dioxide is caused to flow into the gas flow path, and electric discharge is generated between the first electrode and the second electrode. Ammonia is caused to flow into the gas flow path from the ammonia storage source while the electric discharge continues.
(12) In the urea production method, a temperature inside the gas flow path is preferably set to be equal to or higher than a melting point (135° C.) of urea during the electric discharge between the first electrode and the second electrode.

### EMBODIMENTS

### First Embodiment

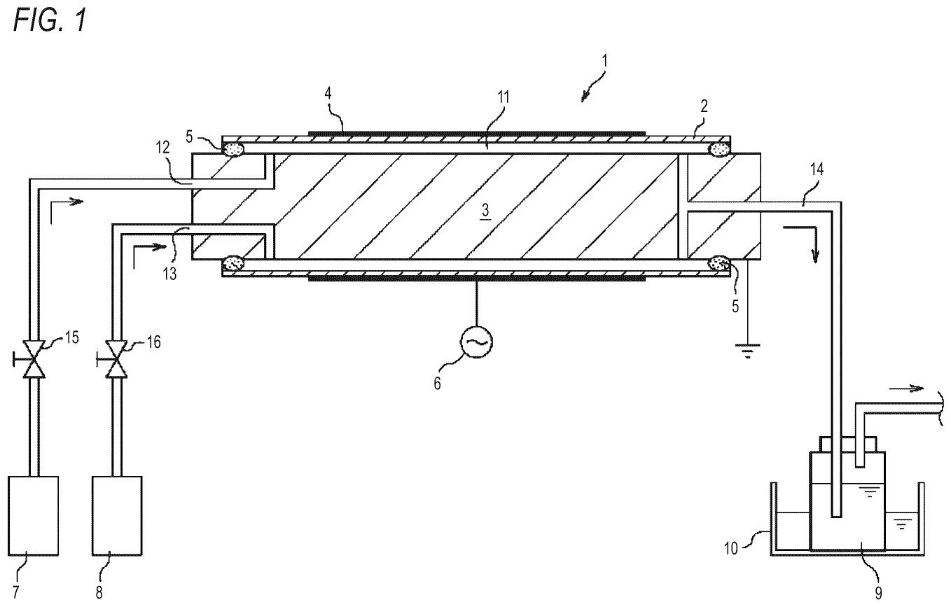

Hereinafter, a preferred first embodiment of the urea production device of the present invention will be described with reference to the drawings. FIG. 1 is a vertical cross-section view schematically showing a urea production device 1 according to the present embodiment. The urea production device 1 includes a body member 2 and sealing members 5 forming a container, a first electrode 3, and a second electrode 4.

The body member 2 according to the present embodiment is made of cylindrical quartz glass (dielectric). The sealing members 5 according to the present embodiment are O-rings made of silicone. The sealing members 5 are disposed inside two end portions of the body member 2 and hold the first electrode 3.

The first electrode 3 is an electrode made of a rod-shaped SUS material having a circular cross section, and an entire length thereof is longer than that of the body member 2. At the two end portions of the first electrode 3, recesses (not shown) for partially accommodating and positioning the sealing members 5 are provided. The first electrode 3 is electrically grounded.

The first electrode 3 is accommodated inside the body member 2 along a central axis of the body member 2 in a state in which the sealing members 5 are engaged with the recesses, thereby being disposed concentrically with respect to the body member 2. The first electrode 3 is fixed in such a manner that the end portions on two sides extend outward from the body member 2. The sealing members 5 close portions between an outer peripheral surface of the first electrode 3 and an inner wall surface of the body member 2 with a predetermined distance therebetween to define a gas flow path 11. The inside of the gas flow path 11 is in a state in which the pressure is not high compared with the related art.

The second electrode 4 is made of a cylindrical SUS material, and is disposed at a position facing the first electrode 3 while being in contact with an outer peripheral surface of the body member 2. That is, the first electrode 3 and the second electrode 4 form at least a pair of electrodes that partially face each other. The second electrode 4 is connected to a power supply 6, and dielectric barrier discharge is generated between the first electrode 3 and the second electrode 4 by applying a voltage. A suitable power supply 6 is a power supply that generates a bipolar pulse waveform and supplies electric power with high electron energy density to the second electrode 4. In the gas flow path 11 between the first electrode 3 and the second electrode 4, a urea synthesis reaction shown in formulas 1 to 3 to be described later occurs. The urea synthesis reaction occurs not only in a region where the first electrode 3 and the second electrode 4 face each other but also in a region of the gas flow path 11 where the first electrode 3 and the second electrode 4 do not face each other.

Two through holes communicating with the gas flow path 11 are formed in one end portion (left end portion in FIG. 1) of the first electrode 3. The two through holes (formed in first electrode 3) form a part of a first raw material introduction path 12 and a part of a second raw material introduction path 13, respectively. Both the two through holes have one side open to a side surface in an axial direction of the first electrode 3, and another side open to a side surface in a radial direction of the first electrode 3. The first raw material introduction path 12 and the second raw material introduction path 13 are formed as different paths (in other words, independent paths) from an inlet to an outlet. A gas raw material in the first raw material introduction path 12 and a gas raw material in the second raw material introduction path 13 are individually supplied to the gas flow path 11 without being mixed. Thus, the synthesis of ammonia and carbon dioxide can be prevented before the ammonia and the carbon dioxide flow into the gas flow path, and the ammonia and the carbon dioxide can be synthesized in the gas flow path.

The first raw material introduction path 12 connects a carbon dioxide storage source 7 and the gas flow path 11, and serves as a carbon dioxide introduction path for supplying carbon dioxide to the gas flow path 11 during operation. The first raw material introduction path 12 in the present embodiment is formed by connecting a pipe extending from the carbon dioxide storage source 7 to the inlet of the through hole of the first electrode 3. A valve 15 is provided in the first raw material introduction path 12 to control a supply amount and supply timing of carbon dioxide.

The second raw material introduction path 13 connects an ammonia storage source 8 and the gas flow path 11, and serves as an ammonia introduction path for introducing ammonia into the gas flow path 11 during operation. The second raw material introduction path 13 in the present embodiment is formed by connecting a pipe extending from the ammonia storage source 8 to the through hole of the first electrode 3. A valve 16 is provided in the second raw material introduction path 13 to control a supply amount and supply timing of ammonia.

The urea production device 1 in the present embodiment includes a gas discharge path 14. The gas discharge path 14 is formed by a through hole which is provided at another end portion of the first electrode 3 (right end portion in FIG. 1) and allows the gas flow path 11 to communicate with the outside, and a pipe which extends from the through hole to the collection part 9. The gas discharge path 14 is used as a flow passage for discharging discharge gas containing raw materials that are not converted into urea, in addition to a flow passage through which the produced urea passes. A collection part 9 is disposed at an outlet of the gas discharge path 14. The collection part 9 can mainly collect the produced urea and ammonia (that is not used for synthesis of urea).

In the present embodiment, the collection part 9 is cooled by a cooling tank 10 to further improve the collection efficiency of urea. In addition, the carbon dioxide discharged from the gas discharge path 14 may be returned to the carbon dioxide storage source 7 via, for example, a filter or the like, or may be collected by some means, although not intentionally shown.

The carbon dioxide storage source 7 that supplies carbon dioxide to the urea production device 1 may be a container that stores carbon dioxide in a solid or gaseous state. The carbon dioxide storage source 7 may be a device for producing carbon dioxide. Similarly, the ammonia storage source 8 may be a container that stores ammonia in a liquid or gaseous state or a device for producing ammonia.

The gas flow path 11 having a distance in an radial direction of 0.1 mm or more and 5 mm or less is formed between the outer peripheral surface of the first electrode 3 and an inner peripheral surface of the body member 2. The outer peripheral surface of the first electrode 3 refers to an outer surface in the radial direction centered on an axial center of the first electrode 3, and the inner peripheral surface of the body member 2 of the container refers to an inner surface in a radial direction centered on an axial center of the container. The distance between the outer peripheral surface of the first electrode 3 and the inner peripheral surface of the body member 2 means a distance in which a radial distance between the outer peripheral surface of the first electrode 3 and the inner peripheral surface of the body member 2 is the shortest in the axial direction.

By setting the distance between the body member 2 and the first electrode 3 to 0.1 mm or more, it is possible to prevent the generation of a urea precursor and to prevent clogging of the gas flow path 11 due to the urea precursor. As a result, it is possible to prevent the production of urea from being stopped.

In addition, by setting the distance between the body member 2 and the first electrode 3 to 5 mm or less, the electric discharge can be uniformly generated in the gas flow path 11. That is, the urea production device 1 in the present embodiment can sufficiently convert the gas raw material (mainly ammonia and carbon dioxide) into plasma, and can prevent a decrease in a production amount of urea.

The temperature inside the gas flow path 11 is raised to 135° C. in 5 to 10 minutes due to Joule heat generated by the electric discharge. That is, the temperature in the gas flow path 11 is lower than that in the related art. In addition, in the present embodiment, the first electrode 3 and the gas discharge path 14 are made of metal having good heat conductivity. Therefore, the urea which receives the heat of the gas flow path 11 and maintains a gaseous state can be discharged to the collection part 9. Therefore, in the gas flow path 11 and the gas discharge path 14, heating by, for example, a heater is not necessary to maintain urea in a gaseous state.

The urea produced by the urea production device 1 according to the present embodiment is generated during the electric discharge in the first electrode 3 and the second electrode 4, and is collected in a solid state from the collection part 9. Regarding the collection of urea, a method may be used in which water is injected from the first raw material introduction path 12 and the second raw material introduction path 13, the solid urea is dissolved in the water, and the urea is discharged from the gas discharge path 14.

A process for producing urea using the urea production device 1 according to the present embodiment will be described. The urea synthesis reaction in the gas flow path of the urea production device 1 is shown in the following formulas 1 to 4.

CO₂+e→CO+0.502+e (Formula 1)

NH₃+e→NH₂+0.5H₂+e (Formula 2)

CO+(2NH₂+H₂)→NH₂CONH₂+H₂ (Formula 3)

H₂+0.5O₂→H₂O (Formula 4)

Formula 1 shows a state in which carbon dioxide is converted into plasma and decomposes into carbon monoxide and oxygen. Formula 2 shows a state in which ammonia is converted into plasma and decomposes into ammonia ions (imide) and hydrogen. Formula 3shows a reaction in which urea is synthesized by reacting carbon monoxide and ammonia ions. Formula 4 shows a reaction in which the oxygen generated in Formula 1 reacts with the hydrogen generated in Formula 3 to generate water.

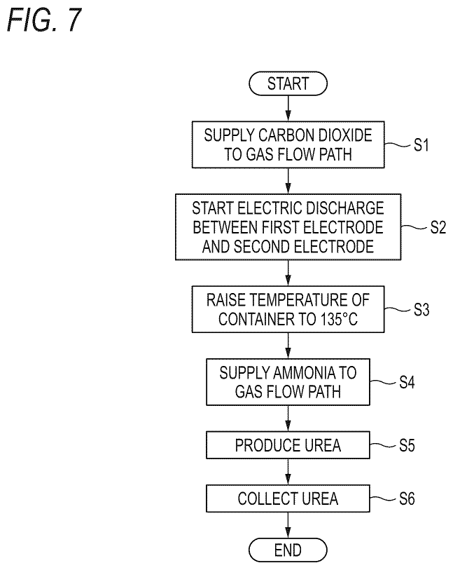

FIG. 7 is a flowchart showing a urea production method using the urea production device 1. Hereinafter, an embodiment of the urea production method will be described with reference to FIG. 7.

First, carbon dioxide is supplied from the carbon dioxide storage source 7 to the gas flow path 11 through the first raw material introduction path 12 (step 1).

Next, in a state in which carbon dioxide is supplied to the gas flow path 11, electric power is supplied to the second electrode 4 to generate electric discharge between the first electrode 3 and the second electrode 4 (step 2). In step 2, a chemical reaction corresponding to the above formula 1 occurs. That is, due to the electric discharge, the carbon dioxide in the gas flow path 11 is converted into plasma and decomposes into carbon monoxide and oxygen. In the present embodiment, as an example, the electric power supplied to the second electrode 4 is set to a voltage of 16 kV and a discharge frequency of 10 KHz.

Further, an ambient temperature of the gas flow path 11 is raised to 135° C. by Joule heat of the first electrode 3 and the second electrode 4 (step 3).

Next, in a state in which the electric discharge between the first electrode 3 and the second electrode 4 is continued, ammonia is introduced into the gas flow path 11 from the ammonia storage source 8 (step 4). In this step 4, due to a reaction corresponding to the above formula 2, the ammonia in the gas flow path 11 is converted into plasma and decomposes into ammonia ions and hydrogen. In Step 4, the chemical reaction shown in formula 1 occurs simultaneously.

Next, a chemical reaction corresponding to formula 3 occurs. In short, urea is produced by a synthesis reaction between the carbon monoxide generated in step 2 and the ammonia ions generated in step 4 (step 5).

Finally, the produced urea is collected by the collection part 9 (step S6).

The urea production device 1 can produce urea by the urea production method using the above steps.

Hereinafter, it will be described in detail that urea can be produced from the carbon dioxide and ammonia converted into plasma by the above steps.

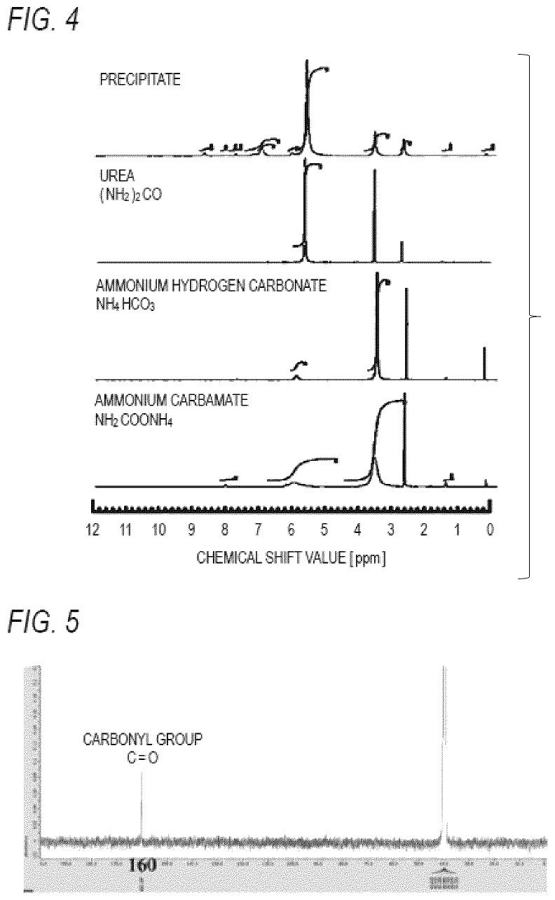

First, as shown in FIGS. 4 and 5, a molar ratio of ammonia to carbon dioxide was set to 1.0, that is, a mixing ratio of ammonia to carbon dioxide was set to 1:1 by volume, and a precipitate produced by converting carbon dioxide and ammonia into plasma were analyzed, as a result, it was confirmed that the precipitate was urea.

FIG. 4 is a diagram showing an analysis result of the produced substance by proton nuclear magnetic resonance. FIG. 5 is a diagram showing an analysis result by Fourier transform infrared ray spectroscopy. As shown in FIG. 4, it was confirmed that the precipitate was highly concentrated urea (partially containing ammonium hydrogen carbonate and ammonium carbamate as precursors). As shown in FIG. 5, the precipitate has a carbonyl group, which is a chemical structure specific to urea, and can be identified as urea.

FIG. 3 shows a relation between an addition ratio of ammonia to carbon dioxide and a conversion rate of carbon dioxide. In combination with the analysis results shown in FIGS. 4 and 5, it was confirmed that when the mixing ratio of ammonia was set to 1:1 (50%:50%) by volume, a conversion rate of carbon dioxide to urea was 33%.

In addition, it was confirmed that the amount of urea produced increased when the addition ratio of ammonia to carbon dioxide described to be later was set to 10% or more. In contrast, when the addition ratio of ammonia to carbon dioxide was set to less than 10%, it was confirmed that ammonium carbamate was generated and the amount of urea produced decreased.

That is, in the present embodiment, as shown in the analysis results shown in FIGS. 3 to 5, urea can be efficiently produced by mixing carbon dioxide with an optimal amount of ammonia and converting the carbon dioxide and the ammonia into plasma.

Further, the urea production method according to the present embodiment controls the introduction timing and molar ratio of carbon dioxide and ammonia, thereby producing urea more efficiently.

First, the “introduction timing” of carbon dioxide and ammonia in the present embodiment will be described.

In general, if a voltage is applied in a state in which both carbon dioxide and ammonia are present in the gas flow path 11, ammonium carbamate as a precursor of urea is generated, and therefore a collection rate of urea decreases.

On the other hand, in the present embodiment, the carbon dioxide becomes urea at a high conversion rate by controlling the introduction timing of carbon dioxide and ammonia and introducing ammonia when carbon dioxide is converted into plasma due to the electric discharge.

Next, the molar ratio of carbon dioxide and ammonia to be introduced will be described.

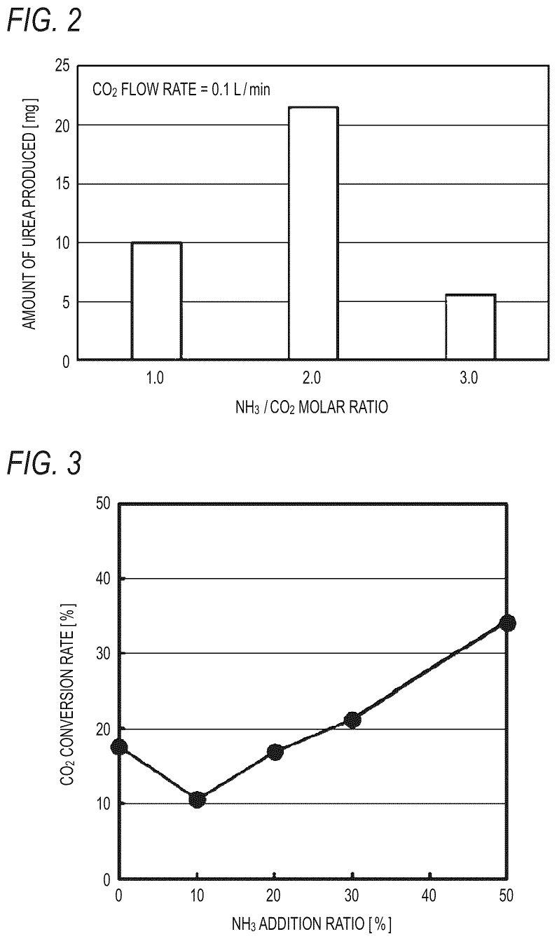

FIG. 2 shows a relation between the molar ratio (1.0 to 3.0) of ammonia and carbon dioxide and the amount of urea produced when flow rates of carbon dioxide and ammonia to be supplied to the urea production device 1 are each set to 0.1 L/min. As compared with a case where the molar ratio of ammonia to carbon dioxide is 1.0 or 3.0, the largest amount of urea can be produced when the molar ratio of ammonia to carbon dioxide is 2.0.

This also coincides with the stoichiometric value (NH₃/CO₂=2.0), and it is clear that urea can be efficiently produced by the direct reaction of carbon monoxide and ammonia ions.

As described above, the urea production device and the urea production method according to the present embodiment can produce urea without applying high-temperature and high-pressure conditions in the production process. The urea production device and the urea production method according to the present embodiment can efficiently produce urea by optimizing the molar ratio of carbon dioxide and ammonia to be introduced.

### Second Embodiment

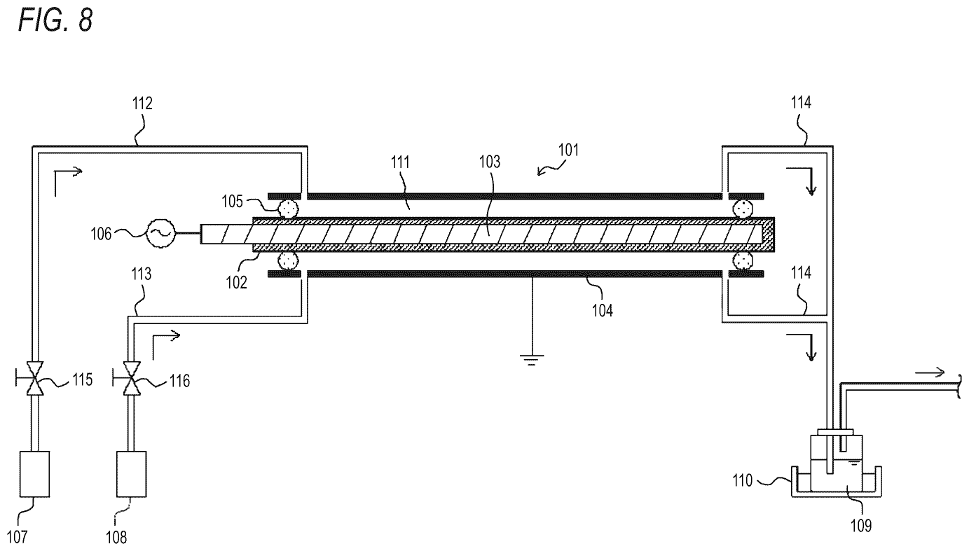

Next, a second embodiment of the urea production device according to the present invention will be described with reference to FIG. 8. FIG. 8 is a vertical cross-section view schematically showing a urea production device 101 according to the present embodiment.

In the second embodiment, when the last two digits of a component number are the same as that in the first embodiment, it means a component having the same function as in the first embodiment.

The urea production device shown in the second embodiment is mainly different from the first embodiment in the position of a gas flow path 111. Specifically, in the present embodiment, the gas flow path 111 is formed between a body member (dielectric) 102 and a second electrode 104. A distance between an inner peripheral surface of the second electrode 104 and an outer peripheral surface of the body member 102 is 0.1 mm or more and 5 mm or less.

The second embodiment is also different from the first embodiment in that two gas discharge paths 114 are formed.

Other configurations in the present embodiment are substantially the same as those of the first embodiment. That is, the urea production device 101 includes the body member 102 including a dielectric, a first electrode 103 disposed inside the body member 102, and the second electrode 104 which is external to the body member 102 and is disposed such that at least a part thereof faces the first electrode 103. The urea production device 101 includes the gas flow path 111 formed between the body member 102 and the second electrode 104, a first raw material introduction path 112 having one end connected to the gas flow path 111 and another end connected to a carbon dioxide storage source 107, and a second raw material introduction path 113 which is a raw material introduction path different from the first raw material introduction path 112 and has one end connected to the gas flow path 111 and another end connected to an ammonia storage source 108. The urea production device 101 can apply a voltage between the first electrode 103 and the second electrode 104 to generate electric discharge.

In addition, the urea production device 101 can be understood as having the following configurations. The urea production device 101 includes a gas introduction path (first raw material introduction path 112 and second raw material introduction path 113) into which a gas flows, the gas discharge paths 114 from which a gas is discharged, and the gas flow path 111 connected to a gas introduction path and the gas discharge paths 114. The urea production device 101 includes the body member 102 including a dielectric and a pair of electrodes (first electrode 103 and second electrode 104) on two sides of the body member 102 such that at least a part of the electrodes faces each other, and the gas flow path 111 is formed between the body member 102 and the second electrode 104. The gas introduction path includes the first raw material introduction path 112 having one side connected to the gas flow path 111 and another side connected to the carbon dioxide storage source 107, and the second raw material introduction path 113 which is a raw material introduction path different from the first raw material introduction path 112 and has one side connected to the gas flow path 111 and another side connected to the ammonia storage source 108. The urea production device 101 can generate electric discharge by applying a voltage between the pair of electrodes (first electrode 103 and second electrode 104).

Further, a urea production method according to the present embodiment is a urea production method in which urea is produced by generating electric discharge between the first electrode 103 disposed inside the body member 102 and the second electrode 104 disposed outside the body member 102 and converting carbon dioxide and ammonia flowing into the gas flow path 111 formed between the body member 102 and the second electrode 104 into plasma.

The urea production method includes a first step of introducing carbon dioxide into the gas flow path 111, a second step of generating electric discharge between the first electrode 103 and the second electrode 104 to convert the carbon dioxide into plasma, a third step of introducing ammonia into the gas flow path 111 while the electric discharge continues, and a fourth step of generating electric discharge to convert the ammonia into plasma.

With the above configurations, the urea production device 101 and the urea production method according to the present embodiment can produce urea under conditions of at a temperature lower than that in the related art and at atmospheric pressure. In short, the urea production device 101 and the urea production method according to the present embodiment can produce urea under conditions that do not require high temperature or high pressure in the production process.

The urea production devices 1 and 101 according to the first and second embodiments can be used under atmospheric pressure, and for example, a pressurizing device or the like for applying pressure to the inside of the urea production devices 1 and 101 is not necessary, and thus the devices can be configured more compactly.

In addition, the urea production devices 1 and 101 according to the first and second embodiments can reduce a discharge amount of carbon dioxide in various processes by being incorporated into another equipment or installed in a discharge path of carbon dioxide.

Further, since the ammonia used in the urea production devices 1 and 101 according to the first and second embodiments uses green ammonia produced in a process in which carbon dioxide is not discharged or blue ammonia produced in a process in which a discharge amount of carbon dioxide is low, a fixed amount of carbon dioxide can be further increased.

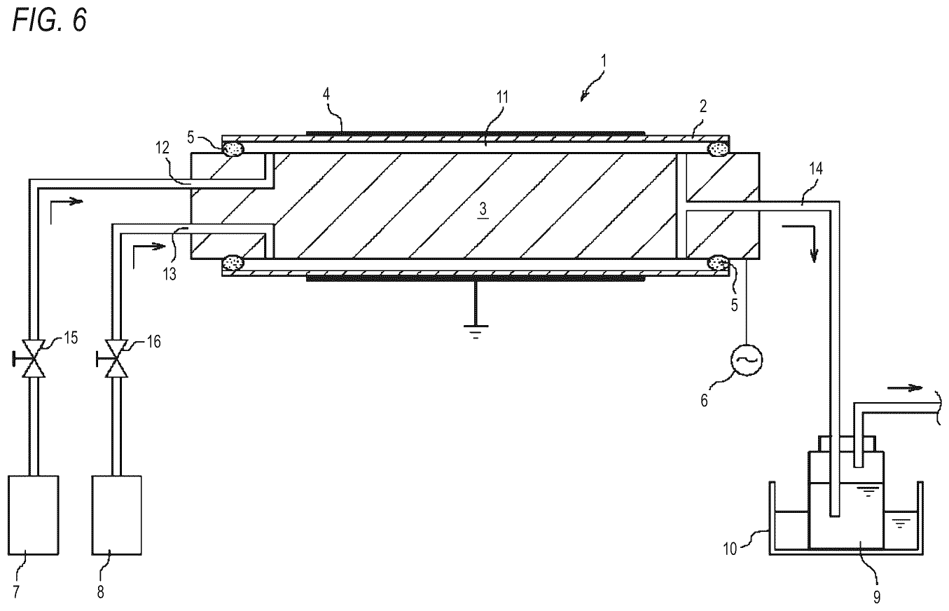

As described above, the technologies related to the urea production device and the urea production method of the present invention have been described above based on the first embodiment and the second embodiment, but the scope of claims is not limited to the embodiments, and the configurations of the urea production device and the urea production method can be appropriately changed. For example, a voltage can be applied to the first electrode. FIG. 6 shows an example of the arrangement of electrodes in an alternative urea production device. In the embodiments, the first electrode is grounded and the second electrode is connected to a power supply to supply electric power, but as shown in FIG. 6, electric power may be supplied to the first electrode and the second electrode may be grounded. In the first raw material introduction path and the second raw material introduction path, the pipes from the carbon dioxide storage source and the ammonia storage source may be directly introduced into the gas flow path without passing through the through hole of the first electrode.

As shown in FIG. 6, an axial length relation of the body member, the first electrode, and the second electrode is such that the first electrode is the longest, and the body member and the second electrode become shorter in this order. However, as long as dielectric barrier discharge can be generated between the first electrode and the second electrode, the axial length relation of the body member, the first electrode, and the second electrode may be limited. For example, the body member (dielectric) may be longer than the first electrode.

The gas flow path may adopt a so-called labyrinth structure in which at least a part of the flow path is meandered in a circumferential direction. With this configuration, it is possible to extend the residence time of the raw material gas under the plasma discharge and to generate a more sufficient synthesis reaction of the raw material gas within a range in which the flow path is not clogged due to the generation of the ammonia precursor.

The first electrode may be divided into a plurality of electrodes, and may be two or more electrodes. In this case, at least a part of each electrode forming the first electrode may face the second electrode. Similarly, the second electrode may also be divided into a plurality of electrodes.

In the first embodiment, one gas discharge path is formed, and in the second embodiment, two gas discharge paths are formed, but the number of the gas discharge path may be one, two, or three or more.

The number of the gas introduction path is not limited as in the case of the gas discharge path. There are a total of two raw material introduction paths, one first raw material introduction path and one second raw material introduction path, but the first raw material introduction path and the second raw material introduction path may be different (independent) flow paths before being input to the gas flow path. For example, a plurality of first raw material introduction paths and a plurality of second raw material introduction paths may be provided, and two or more first raw material introduction paths and two or more second raw material introduction paths may be formed.

In the first embodiment and the second embodiment, urea is produced using the urea production device under atmospheric pressure condition, but a pressure may be applied to the urea production device (more specifically, gas flow path) within a range in which the pressure is not high.

In the first embodiment and the second embodiment, the gas discharge path made of metal having good heat conductivity is used, but other materials may be used. In order to maintain the state of the gas such as urea to be discharged, a heat retaining member or a heating member may be disposed in the gas discharge path.

The body member of the first embodiment is formed of only a dielectric, but may be formed of a combination of a dielectric and other members. However, in this case, a region of the body member where the first electrode and the second electrode face each other is formed of a dielectric.

What is claimed is:

1. A urea production device comprising:
a body portion including a dielectric;
a first electrode disposed inside the body portion; and
a second electrode which is external to the body portion and which is disposed such that at least a part thereof faces the first electrode, wherein
the urea production device includes
a gas flow path formed between the body portion and either the first electrode or the second electrode,
a first raw material introduction path having one side connected to the gas flow path and another side connected to a carbon dioxide storage source, and
a second raw material introduction path which is a raw material introduction path different from the first raw material introduction path and which has one side connected to the gas flow path and another side connected to an ammonia storage source, and
a voltage is to be applied between the first electrode and the second electrode and electric discharge can be generated.

2. The urea production device according to claim 1, wherein
The second raw material introduction path is formed as a different path from the first raw material introduction path, from an inlet to an outlet.

3. The urea production device according to claim 1, wherein
the gas flow path is formed between the body portion and the first electrode, and
a distance between an outer peripheral surface of the first electrode and an inner peripheral surface of the body portion is 0.1 mm or more and 5 mm or less.

4. The urea production device according to claim 1, wherein
the gas flow path is formed between the body portion and the second electrode, and
a distance between an inner peripheral surface of the second electrode and an outer peripheral surface of the body portion is 0.1 mm or more and 5 mm or less.

5. The urea production device according to claim 1, wherein
the first raw material introduction path and the second raw material introduction path are connected to one end portion of the gas flow path, and another end portion of the gas flow path is provided with a collection part configured to collect urea produced by a synthesis reaction of ammonia and carbon dioxide due to the electric discharge.

6. A urea production method in which urea is produced by generating electric discharge between a first electrode disposed inside a body portion including a dielectric and a second electrode disposed outside the body portion and converting carbon dioxide and ammonia flowing into a gas flow path formed between the body portion and either the first electrode and the second electrode into plasma, the urea production method comprising:
a step of introducing the carbon dioxide into the gas flow path;
a step of converting the carbon dioxide into the plasma by generating the electric discharge between the first electrode and the second electrode;
a step of introducing the ammonia into the gas flow path while the electric discharge continues; and
a step of converting the ammonia into the plasma by generating the electric discharge.

7. The urea production method according to claim 6, wherein
the step of introducing the carbon dioxide into the gas flow path is conducted from a first raw material introduction path, and
the step of introducing the ammonia into the gas flow path is conducted from a second raw material introduction path, the second raw material introduction path being formed as a different path from the first raw material introduction path, from an inlet to an outlet.

8. The urea production method according to claim 6, wherein
a molar ratio of the carbon dioxide and the ammonia to be introduced into the gas flow path is the carbon dioxide:the ammonia=1:1 or more and 1:3 or less.
